(19) United States
(12) Patent Application Publication
(10) Pub. No.: US 2014/0212870 A1
(43) Pub. Date: Jul. 31, 2014

(54) FET Sensors With Subtractive Probes for Indirect Detection and Methods

(71) Applicant: Krutarth Trivedi, Plano, TX (US)

(72) Inventor: Krutarth Trivedi, Plano, TX (US)

(21) Appl. No.: 13/947,017

(22) Filed: Jul. 19, 2013

Related U.S. Application Data
(60) Provisional application No. 61/673,541, filed on Jul. 19, 2012.

(51) Int. Cl. G01N 33/543

(52) U.S. Cl. CPC G01N 33/54373
USPC 435/6.11; 435/287.2

(57) ABSTRACT

The present invention relates to compositions on a FET sensor for detecting wide variety of biological entities. The composition of the FET sensor comprises a linker probe having a region for binding a biological entity, and enzymatic region that can cleave or change the position of a cargo molecule bound to the linker probe. The binding of the biological entity may cause a first strand of DNA to dehybridize from a second strand of DNA resulting in a change in conductance of the FET sensor. When the conformation of the probe changes, the conductance of the FET changes. This method provides an advantage over the conventional FET biosensors that use antibodies as probes since the size of nucleotide aptamer probes is smaller, their conformation/shape is well controlled, and their charge is fixed for a wider range of solution conditions, enabling robust detection of target entities with high sensitivity and specificity.

### CROSS-REFERENCE TO RELATED APPLICATIONS

This application claims the benefit of U.S. Provisional Application No. 61/673,541, filed on Jul. 19, 2012.

### FIELD OF THE INVENTION

This invention relates to the field of biosensors. More particularly, this invention relates to a Field Effect Transistor (FET) biosensor for molecular detection using a bound linker probe having an aptamer for binding a target molecule.

### BACKGROUND OF THE INVENTION

Detection of target entities, such as small molecules, oligonucleotides or proteins, is typically accomplished by electrochemical or optical techniques. Despite the simple structure and compact form factor of modern electrochemical sensors such as blood glucose sensing strips, low sensitivity and poor selectivity make detection of bio-entities like protein and DNA difficult. Due to this limitation, application of electrochemical sensors in molecular diagnostics has been constrained. As a result, optical methods, such as, for example, Enzyme Linked Immunoassay (ELISA), are currently the gold standard for detection of bio-entities. However, the overall complexity and high cost, limited sensitivity, and more importantly, lack of portability, make utilization of such methods for point-of-care applications difficult. Alternatively, semiconductor field effect transistor (FET) based sensors offer rapid, low-cost and direct detection of a variety of target entities with high sensitivity and specificity. Ion-sensitive field effect transistors (ISFETs) are an example of the earliest semiconductor devices designed to measure variation in surface charge on the exposed gate dielectric of a FET. The surface of the gate dielectric can be modified with linker probe molecules for capture of specific targets, such as proteins or oligonucleotides, which carry a net charge. The charge of the captured target molecule then causes a proportional change in conductance of the FET sensor. However, for an appreciable change in conductance to occur, the net charge of the captured entities must be sufficiently large and close to the surface, within the thin electric double layer (Debye screening region), so as not to be screened by bulk solution. The charge of the captured entities can vary with size, shape, number of charged groups/residues, pH, and binding efficiency, while electric double layer thickness or Debye length can vary with salt concentration. Therefore, repeatable detection of captured entities, with adequate signal, is difficult when relying directly on the charge of the captured entities to induce appreciable change in conductance of a FET sensor.

U.S. patent application Ser. No. 11/438,758 to Chasin et al., incorporated herein by reference in its entirety, teaches a nucleic acid aptamer-based linker probe molecules that can detect the presence of specific target entities or target substances such as ions, enzymes, proteins, viruses, small molecules, bacteria and provide an amplified response to the detection as manifested by the release of enzymes, reporter signals or drugs. The detection and response is based on nucleic acid functionalities, such as aptamer regions that are designed to specifically bind to almost any entity or ligand, coupled to enzymatic regions that can cleave nucleic acids at specific sequences. However, measuring the concentration or presence of target molecules is still difficult, with even with current technologies that use aptamer-based linker probes because the detection methods lack of high sensitivity and specificity and are affected by the characteristics of target molecule. Therefore, devices that can measure extremely small quantities of target molecules that are not affected by the characteristics of the solution that the target molecules are in, are still needed.

### SUMMARY OF THE INVENTION

The invention allows for highly sensitive and specific detection of a wide variety of target entities, independent of any of their individual characteristics and properties, and potentially enables embodiment FET biosensors to detect target entities such as ligands, ions, or other biospecies in undiluted physiological samples. In particular, this method allows embodiment FET biosensors to detect small molecules, having low net charge, with high sensitivity. Using embodiment FET biosensors to detect biospecies also provides an advantage over the conventional practice of FET biosensors, which use antibodies as probes. The use of probes that are comprised of DNAzymes/aptamers/ssDNA is advantageous because the size of these probes are smaller than antibody probes, their conformation/shape is well controlled, and their charge is fixed for a wider range of solution conditions. This approach of using the DNAzyme/aptamer/ssDNA probes effectively decouples FET sensor response from the physical properties of the target entity and the solution, enabling robust and repeatable quantitative detection of target entities with highest sensitivity and specificity.

In one aspect of the invention a field effect transistor (FET) biosensor comprises a field effect transistor having a FET gate dielectric surface and a linker probe attached to said FET gate dielectric surface. The linker probe has an enzymatic region capable of cleaving nucleic acids having a predetermined nucleic acid sequence and an aptamer region attached to said enzymatic region, said aptamer region capable of selectively binding a target entity. The binding of the target entity to the aptamer induces a measurable change in an electrical parameter of said FET.

In another aspect of the invention the field effect transistor (FET) biosensor has a FET gate dielectric surface. A linker probe is bound to the FET gate dielectric surface. The linker probe comprises a first region defined as a stump region or segment that is attached to the gate dielectric surface, and a second region, defined as a sacrificial region or segment, bound to the stump region. The sacrificial region is capable of detaching from the stump region in the presence of a target molecule, such as a ligand. When the target molecule binds to a specific binding site such as an aptamer on the linker probe, the linker probe releases the sacrificial region, which changes the charge of the linker probe. The change in charge of the linker probe can be measured via a change in an electrical parameter such as the conductance of the FET sensor. In one aspect of the invention, the charge is carried by a cargo molecule (charge carrier) and the linker probe is comprised of nucleotides, such as DNA or RNA.

In another aspect of the invention, the linker probe comprises a DNAzyme or ribozyme that cleaves a nucleic acid sequence when a target molecule binds to a binding site such as an aptamer specifically engineered to hybridize with specificity. When the target molecule binds to the aptamer, the DNAzyme or ribozyme is activated and cleaves the linker probe into the stump segment and the sacrificial segment, leaving only the stump segment bound to the surface of the FET gate dielectric. The sacrificial segment is released into the solution away from the FET gate dielectric surface. Since stump segment imparts a different measurable charge to the FET surface than when the linker probe comprises both the stump segment and sacrificial segment both bound as the link probe, the presence or concentration of the target molecule can be determined by measuring a baseline electrical parameter such as conductance or drive current before target molecule solution is added, and then taking a second measurement of the electrical parameter after target molecular solution is added to the FET.

In yet another aspect of the invention, the FET is an ion-sensitive field effect transistor, a bio-FET, a nanowire FET or a bio-finFET.

In yet another aspect of the invention, the linker probe, which may be comprised of an aptamer, an enzymatic region (such as a DNAzyme or ribozyme), and a sacrificial region is covalently attached to the FET channel surface. In addition, the sacrificial region which may be a charge packet attached to one end of the linker probe, is cleaved off from the linker probe when the target molecule binds to the aptamer. The release of the charged packet, imparts a great difference in charge to the FET compared to when the charged packet is attached to the linker probe when the target molecule is not bound to the aptamer.

In yet another aspect of the invention, the linker probe can be a double stranded piece of DNA having a charge packet at one end of the linker probe. In the absence of a target molecule, the charged packet is near the channel surface of the FET due to hybridization of two strands of DNA. However, in the presence of a target molecule, the target molecule binds to one strand of the DNA, and dehybridizes the second strand of DNA from the first strand of DNA. This dehybridization causes the charge packet to either 1) detach from the DNA molecule, or 2) tether away from the surface of the FET gate dielectric. In either situation, the amount of charge near the FET surface is reduced in the presence of the target molecule, and this difference in charge near the gate dielectric can be measured via the FET to determine the presence or concentration of the target molecule.

In yet another aspect of the invention, a field effect transistor biosensor comprises a field effect transistor having a FET gate dielectric surface and a linker probe attached to said FET gate dielectric surface. The linker probe has a conformation changing region capable of changing three dimensional shape in the presence of a target entity and an aptamer region attached to said conformation changing region, the aptamer region capable of selectively binding a target entity. The binding of the target entity to the aptamer region results in a conformational change of the conformation changing region, thereby inducing a measurable change in an electrical parameter of the FET.

In another aspect of the invention there is a method of indirectly detecting the presence of or concentration of a target molecule with a FET sensor. The method comprises the steps of taking a first measurement of an electrical parameter of an FET to determine a baseline of said electrical parameter. The FET can be any embodiment of an FET described above as aspects of the invention. A next step is placing a solution having an unknown quantity of said target entity in contact with the linker probe on said gate dielectric surface. Then a second measurement of the electrical parameter is taken with the FET. The user determining the presence or concentration of the target molecule by comparing said first measurement of said electrical parameter with said second measurement of said electrical parameter. If the first measurement is different from said second measurement by a threshold value, the target molecule is determined to be present in the solution; and the greater the difference between said first measurement and said second measurement of said electrical parameter, the higher the concentration of said target molecule in the solution. A calibration curve can be used to determine the amount of a target entity by comparing the results of the electrical parameter differences to known concentrations of a solution having a target entity.

The above aspects of the invention, such as the location of the aptamer, DNAzyme or ribozyme, charge carrier, and/or which end of the linker probe is attached to the FET gate dielectric surface, can be combined in various permutations to form several embodiments of the same inventive concept of having a specific aptamer that hybridizes a target molecule with specificity, which then changes the charge of a linker probe that can be measured on an FET sensor.

### BRIEF DESCRIPTION OF THE DRAWINGS

These and other features and advantages of the present invention will become appreciated as the same becomes better understood with reference to the specification, claims and drawings wherein:

FIG. 1 is an embodiment of a linker probe molecule having an aptamer, enzymatic region, and cargo region.

FIGS. 2A-2D illustrate the steps of a linker probe releasing a cargo region after a target molecule binds with an aptamer.

FIGS. 3A-C illustrate the steps of a double stranded DNA molecule releasing bound messenger molecules after binding a target molecule to the DNA molecule.

FIGS. 4A-C illustrate the steps on a FET sensor of an aptamer and DNAzyme released from the cargo region bound to the FET sensor when a target molecule binds to the aptamer region of a linker probe.

FIGS. 5A-C illustrate the steps on an FET sensor of the cargo region of a linker probe being released from the aptamer and DNAzyme regions, when a target molecule binds to the aptamer region of the linker probe.

FIGS. 6A-C illustrate the steps of a releasing a charged cargo region from a gate dielectric surface after a target molecule binds to the aptamer region of the linker probe and dehybridizes the double stranded DNA of the linker probe.

FIGS. 7A-B illustrate the release and/or conformational change of positive charges near an FET gate dielectric surface after a target molecule binds to the single stranded hair-pin loop structure of a DNA linker probe.

### DETAILED DESCRIPTION OF THE EMBODIMENTS

It is to be understood that this disclosure is not limited to the particular embodiments described. It is also to be understood that the terminology used is for the purpose of describing particular embodiments only, and is not intended to be limiting, since the scope of the present disclosure will be limited only by the appended claims.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

It must be noted that, as used in the specification and the appended claims, the singular forms “a,” “an,” and “the” include the plural references unless the context clearly dictates otherwise.

Unless defined otherwise, all terms used herein have the same meaning as commonly understood by one of ordinary skill in the art of which this disclosure belongs. Although any methods and material similar or equivalent to those described herein can also be used in the practice or testing of the present disclosure, the preferred methods and materials are now described.

This invention discloses a unique method for specific and sensitive detection of target entities, in which, change in conductance of the FET sensor is proportional to concentration of captured targets, but is not determined directly by charge of the captured species, rather it is determined by the change in the charge of a linker probe molecule when it captures a target molecule. As described by the figures, the surface of the FET sensor is modified with a linker probe molecule designed to specifically capture target species.

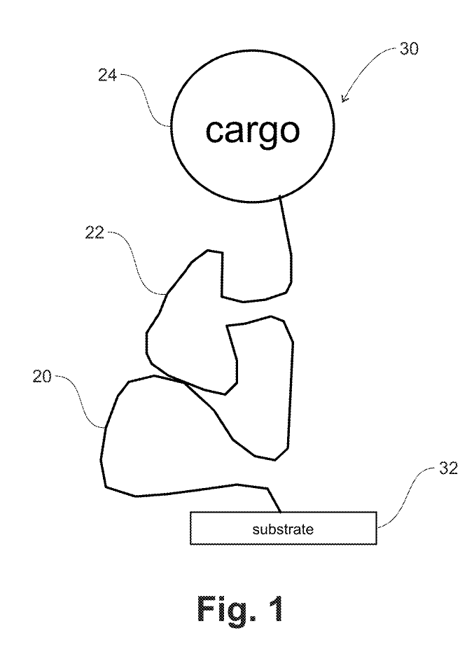

A general example linker probe molecule is illustrated in FIG. 1. The linker probe molecule 30 is attached to the gate dielectric on the channel region of a field effect transistor (FET). FET technologies are commonly known in the art, and are taught in U.S. patent application Ser. No. 13/590,597, to Wu et al., PCT Application Publication No. WO 2012/050873 to Hu et al., U.S. Pat. No. 8,262,900 to Rothberg et al., U.S. patent application Ser. No. 11/033,046, U.S. Pat. No. 7,303,875 to Bock et al., and “Ion-Sensitive Field Effect Transistor for Biological Sensing” Sensors (2009), vol. 9, pages 7111-7131, all incorporated herein by reference in their entireties. The example linker probe molecule comprises an aptamer region 20, an enzymatic region 22 and a cargo region 24. The nucleic acid aptamer regions 20 typically range from about 15 to 500 nucleotides and can bind to almost any molecular or macromolecular entity such as ligands, ions, small organic molecules, nucleic acids, proteins, fungi, and bacteria cells. Aptamers are created and selected using a combination of synthetic chemistry, enzymology, and affinity chromatography and are single-stranded or double stranded oligonucleotides that bind to a particular ligand with great affinity and selectivity. The aptamer region 20 can have an enormous variety of shapes due to the number of possible combinations of a sequence of four different nucleic acids. For example, the chemical synthesis of an oligonucleotide that incorporates a sequence of 25 nucleotides that are randomly selected from the 4 possible DNA bases results in a population of 10¹⁵ different molecules of unique sequence and diverse 3-dimensional conformations. Because there are so many different chemical identities in such a population, it is possible to find a sub-population of these oligonucleotides that exhibit an affinity to almost any chemical structure. These ligand-binding nucleic acid molecules are the aptamers that are then incorporated into the aptamer region 20 of the linker probe 30. After a specific aptamer is found that binds specifically to the target entity (such as a ligand, ion, small organic molecule, nucleic acid, protein, fungi, bacterial cells, etc.), the aptamer is used to bind those substances, and the binding of the substance to the aptamer region 20 can be used to directly and indirectly detect the presence of these substances.

In addition to the aptamer region, the linker probe can also include other regions to impart specific features that aid in the detection of molecules. One such region that may be incorporated is an enzymatic region, such as a ribozyme or DNAzyme. One type of linker probe can incorporate a ribozyme. Ribozymes are RNA molecules that are capable of the sequence-specific cleaving of mRNA molecules. Another type of linker probe can incorporate DNAzymes, which are analogs of ribozymes, but with greater biological stability. Ribozymes or DNAzymes can be created and incorporated within the linker probe such that the ribozymes and DNAzymes cleave only at a specific nucleotide recognition sequence on the linker probe. The cleaving region may be designed to remain inactive (that is, remain intact) until the linker probe 30 binds with the target entity (ligand) 34 and undergoes a ligand-aptamer dependent conformational change.

When a target molecule 34 binds to the aptamer region 20, a conformational change in the enzymatic region 22 causes the region 22 to become activated. When activated, the enzymatic region 22 may cause the cargo region 24 to be cleaved from the linker probe 30. If the cargo molecule 24 carries significant charge, the conductance in channel of the FET 32 when the cargo molecule 24 separates from the linker probe molecule. The change in conductance is proportional to the number of cargo molecules that are cleaved and is directly proportional to the concentration of the target molecule.

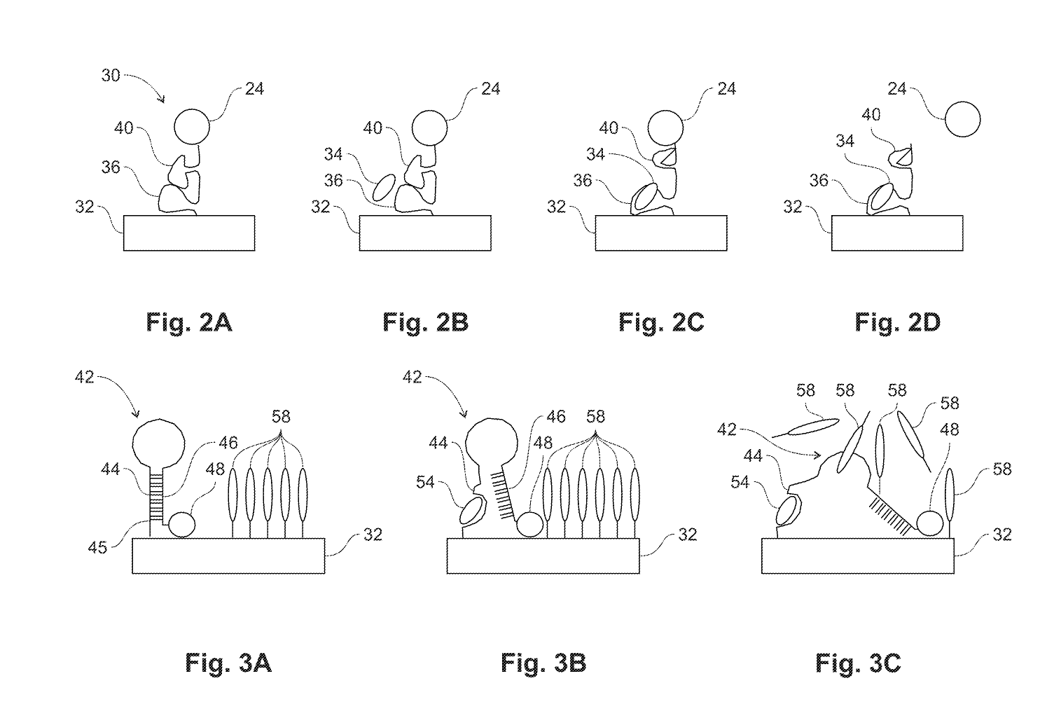

FIGS. 2A-2D illustrate one embodiment with a linker probe 30 attached to a FET channel substrate 32 where a target entity 34 binds to an aptamer region 36 and releases the cargo region 24. FIG. 2A illustrates the linker probe 30 with no target entity 34 present. In FIG. 2B, a target entity 34 approaches the aptamer region 36 of the linker probe 30. The aptamer region 36 is designed to bind to the target entity 34 with great selectivity and specificity such that it will only bind to the target entity 34 and not bind to other molecules that may exist in the solution. In FIG. 2C the target entity 34 binds with the aptamer region 36 causing a conformational change in the DNAzyme region 40 of the linker probe 30. In FIG. 2D the conformational change activates the DNAzyme (such as by positioning the DNAzyme near the recognition sequence), causing the DNAzyme to cleave a specific nucleotide recognition sequence close to the cargo region 24, releasing the cargo region 24 into solution. The DNAzyme 40 alternatively can be any region that is characterized by enzymatic activity, such as a ribozyme. In an embodiment where the substrate 32 is an ion sensitive FET and the cargo region 24 is a charge packet which carries a significant amount of charge. When the charge packet 24 is released from the linker probe, the ion sensitive FET detects a change in conductance caused by the change in the charge of the linker probe due to the release of the cargo region charge packet 24.

FIG. 3A illustrates another type of linker probe 42. Here, the linker probe comprises a first strand 44 and a second strand 46 of a double-stranded DNA molecule. The DNA strand on the left 44 may be an aptamer designed to bind, with selectivity and specificity, to a target entity 54. The length and base pair density of the DNA strand on the left 54 is designed such that the DNA sequence binds (hybridizes) to the DNA strand on the right 46 less strongly than it does the target entity 54. Also attached to the substrate 32 are messenger molecules 58 which can be designed to carry significant charge. When the substrate 32 is the channel of an ion sensitive FET and the messenger molecules 58 are cleaved from the surface, the ion sensitive FET detects a change in conductance caused by the change in the surface charge. In FIG. 3A, the linker probe 42 is bound to the ion sensitive FET 32 and no target entity is in the solution. The messenger molecules 58 are also bound to the substrate and provide a baseline measuring signal. As shown in FIG. 3B when a target entity 54 binds to the aptamer 44, a conformation change occurs on the linker probe 42, causing release of the second DNA strand 46 from the first DNA strand 44, allowing the cargo molecule 48 to extend away from the aptamer 44. Referring now to FIG. 3C, the cargo molecule 48 which may be an enzyme, remains tethered to the linker probe 42, but is free to move along the surface of the ion sensitive FET to catalyze the release of the messenger molecules 58 from the surface of the FET. As illustrated in FIG. 3C, several messenger molecules 58 can be released (and thus measured by the change in charge) with the binding of only a single target entity 54, thereby allowing amplification of the target entity signal since a single bound target molecule 54 can release several messenger molecules 58, thereby significantly changing the measured conductance of the FET.

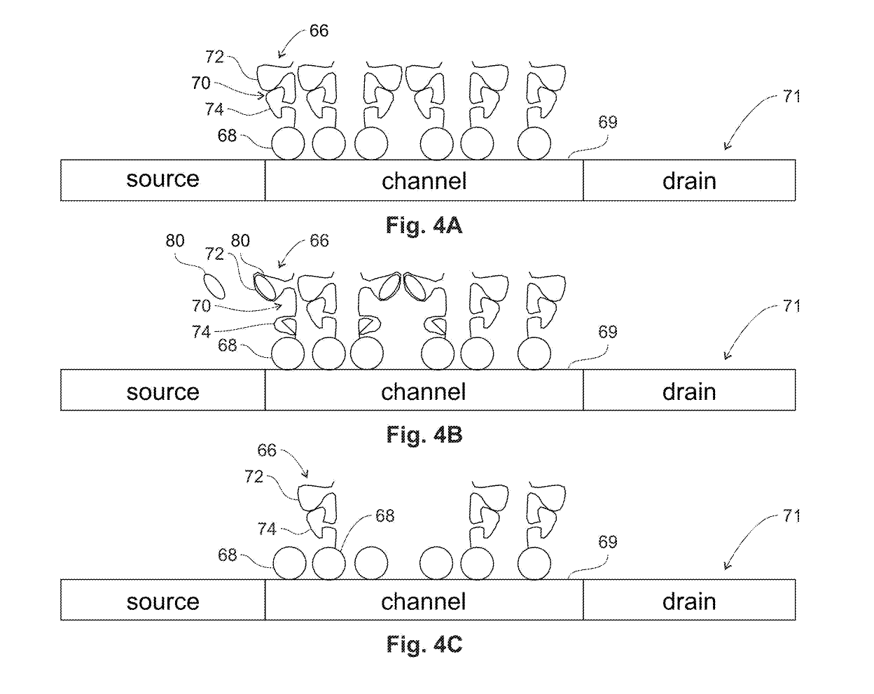

In one embodiment, illustrated in FIGS. 4A-4C, the linker probe 66 is bound to the channel region surface 69 of a FET 71. Here, the linker probe 66 is composed of at least two defined regions, first region is a “stump molecule” or region 68 and, and a second region is a “sacrificial molecule” or region 70. The stump molecule 68 has a fixed charge or may be a molecule containing little or no charge. The stump molecule 68 remains bound to the surface of the sensor 69 and the “sacrificial molecule” 70, which is designed to cleave or release and detach when a target entity 80 binds to the aptamer 72. In this embodiment the sacrificial molecule 70 may be comprised of an aptamer region 72 which selectively binds to the target entity 80, and a DNAzyme region 74 which is activated when a target entity 80 binds to the aptamer 72. The surface coverage of the linker probe 66 can be well controlled, and the conductance before capture of a target entity 80 may be characterized and calibrated as a baseline conductance. In this embodiment, the sacrificial molecule 70 may be designed to have significant charge, so as to specifically cause a larger change in conductance upon detachment from the stump molecule 68. In this manner a target molecule with little or no charge may cause the sacrificial molecule to be cleaved from the stump molecule resulting in a large change in conductance of the FET.

As shown in FIG. 4B, when a target entity 80 binds to the aptamer region 72, the DNAzyme region 74 of the sacrificial molecule 70 may undergo a 3-D conformational change that activates the DNAzyme, such as by positioning the DNAzyme near the cleavage sequence, thereby releasing the sacrificial molecule 70 from the stump molecule 68.

Referring now to FIGS. 4B and 4C together, the activated DNAzyme 74 cleaves the sacrificial molecule 70 from the stump portion 68 of the linker probe 66, leaving only the stump molecule 68 attached to the FET channel surface 69 of the FET. The sacrificial molecule 70 may be a charge packet constructed to carry significant charge and as the sacrificial molecule 70 detaches and moves away from the gate surface 69 of the FET channel. When this occurs, total charge on gate surface 69 of the FET channel within the electric double layer is changed, causing a marked and repeatable change in conductance of the FET sensor. Advantageously, the conductance change, or signal of the FET sensor 71, results primarily from detachment of the sacrificial molecule 68 of fixed charge and is, therefore, independent of the properties of the target entities 80 and their interactions with solution. Additionally, the baseline signal of the FET sensor 71 is calibrated with the full charge of un-cleaved/un-released linker probe, which is closer to the surface and may be smaller in size, as compared to the target entities 80 to be captured. As a result, the total change in surface charge upon capture of target entity 80 is always due to a subtractive change on the surface (surface loses fixed charge). As the change in molecules, as well as charge, on the surface is subtractive, the double layer or Debye length cannot mask the resulting signal, making it insensitive to solution conditions such as salt concentration, physical properties of the target entity 80 such as net charge, its location on the entity, and confirmation of captured entity, resulting in improved robustness and reliability of the detection method.

As shown in FIG. 4A, the dielectric over the FET channel surface 69 of the FET biosensor 71 may be covered with many linker probes 66. Then, as shown in FIG. 4B, when the channel of the FET biosensor 71 is immersed in a sample solution containing an unknown concentration of target entities 80, if there is a low concentration of target entities 80, few will be captured by the binding region 72 of the linker probe, but if there is a high concentration of target entities 80, many target entities 80 will be captured by the binding regions 72 on several the linker probes 66. As shown in FIG. 4C, if few target entities 80 are captured by the linker probe 66, then few of the linker probes will cleave/release, causing the charge packet 68 with little or no charge to remain attached to the dielectric of the FET biosensor 71 and causing the sacrificial molecule 70 having significant charge to diffuse away into solution, whereas if many target entities are bound to the binding region 72 of the linker probe 66, then many will cleave/release and many sacrificial molecules 70 will diffuse away into solution. In this manner, the change in charge caused by the cleaving/release of the sacrificial molecule 70 may be directly correlated to the concentration of target entities 80 in solution, which can be measured by a change of conductance of the FET 71.

In a method of assaying the target sample, the FET biosensor 71 may first be biased into the subthreshold region where a linear change in charge on the gate 69 causes a logarithmic change in channel current for maximum sensitivity. An electrode may be immersed in the sample solution to affect the biasing or in the case of a fin-FET biosensor, the substrate under the box oxide may be used to bias the biofin-FET into the linear region.

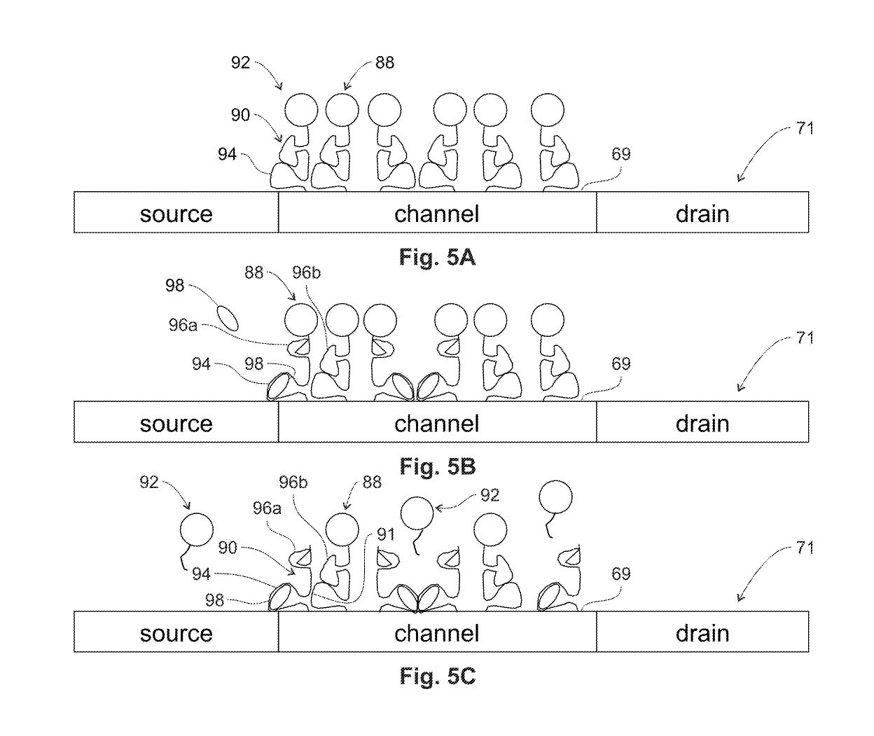

Another embodiment of using linker probes on an FET to measure the presence of target molecules is illustrated in FIGS. 5A-5C. The embodiment has the reverse arrangement of the stump molecule 68 and sacrificial molecule 70 of FIGS. 4A-C. In FIGS. 5A-5C, the linker probe 88 also comprises a sacrificial molecule 92 and stump molecule 90. The stump molecule 90 comprises an aptamer region 94 which binds to a target entity 98 with high selectivity and specificity, and a DNAzyme portion 96 which under goes a conformational change from an inactive state 96b to an active state 96a when a target entity 98 binds to the aptamer 94. The stump molecule 90 is attached to the gate dielectric 69 over the channel region of the FET biosensor 71. The sacrificial molecule 92 may be a charged packet that carries significant charge such as a protein or a polymer containing many acid or base groups. Each region of the linker probe 88 may have a well-defined electronic charge. The linker probe 88 has a first charge when the stump molecule 90 and sacrificial molecule 92 are linked together and also bound to the FET channel surface 69. This charge determines the baseline conductance of the FET sensor 71, but when the stump molecule 90 and sacrificial molecule 92 are detached, the stump portion has different charge, which changes the conductance of the FET sensor 71.

As shown in FIG. 5B, when a target entity 98 binds to the aptamer region 94 of the stump molecule 90, the DNAzyme 96 may undergo a 3-D conformational change, where the DNAzyme may be configured in a first inactive conformation 96b and change into a second activated conformation 96a, which cleaves off the sacrificial molecule 92, thereby changing the conductance of the FET sensor 71.

As shown in FIG. 5C the activated DNAzyme 96a cleaves the sacrificial molecule 92, leaving the stump molecule 90 on the surface of the sensor. This capture and cleavage event causes the remaining probe molecule to have a reduced charge because the positive charge on the sacrificial molecule 92 is no longer bound the stump molecule 90, which is bound to the channel surface 69 of the FET 71. The release of the sacrificial molecule 92 therefore leads to a significant measurable change in conductance of the FET sensor 71, thereby allowing the user to measure the concentration of target entity 98.

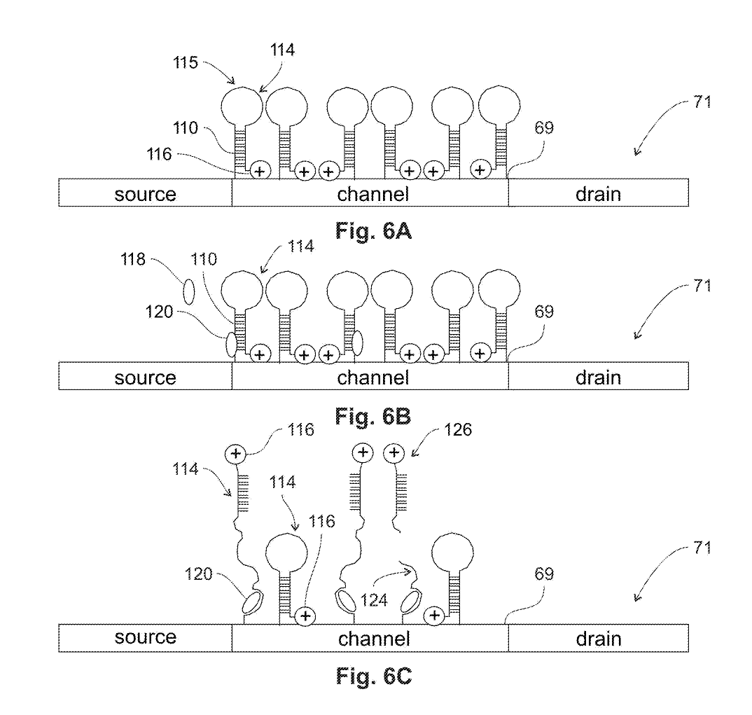

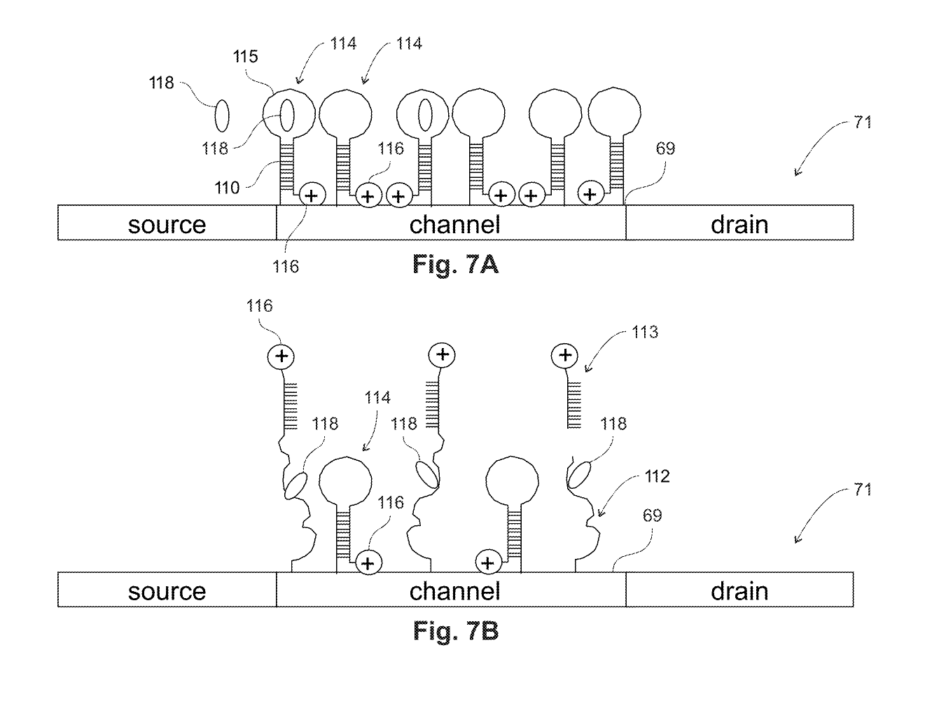

In other embodiments, such as those is illustrated in FIGS. 6 and 7, the linker probe 114 comprises two linked regions, an oligonucleotide 110 and a charge packet cargo region 116, coupled together via a molecular tether 115, or may be separated regions. The oligonucleotide region 110 may be an aptamer, a DNAzyme, a ribozyme, or an enzyme molecule, while the molecular tether 115 may be an aptamer, a DNAzyme, a ribozyme, an enzyme molecule, or polymer. In the first detection method, the target molecule 118 binds to the oligonucleotide 110, as shown in FIG. 6B, causing its dehybridization of one DNA strand from a second DNA of the oligonucleotide region 110, illustrated in FIG. 6C. In one scenario, upon dehybridization, the dehybridized strands remain linked together by the molecular tether 115. In another scenario, without the molecular tether, upon dehybridization the untethered strand 126 diffuses away into solution. When the targeting entity 118 binds to the oligonucleotide 110, the linker probe releases the charged packet 116 away from the FET channel surface 69. In another scenario, the molecular tether may contain a DNAzyme or enzyme and may cleave after binding of the target entity 118, forming a bound segment (i.e. a stump molecule or region) 124 to the channel, and a released dehybridized segment 126 into solution. The charge packet 116 which carries significant charge (either positive or negative) such as a protein, chelate, or polymer containing acid or base groups significantly changes the measurable charge on the FET, and when the charge packet 116 is released from the surface of the channel 69, the FET detects the charge difference, thereby measuring the concentration of target entity 118 in solution.

In another embodiment, shown in FIG. 7A, the molecular tether 115 is an oligonucleotide, such as an aptamer, that captures the target molecule 118, causing dehybridization of the attached double stranded oligonucleotide 110, as shown in FIG. 7B. Each region of the linker probe 114 has a well-defined electronic charge when the charge packet 116 and oligonucleotide 110 are bound to the channel, which determines the baseline conductance of the FET sensor 71. In a similar detection method to the previously described method illustrated in FIG. 6, the capture event may or may not cause cleavage of the molecular tether 115. Here, the charge packet 116 may be tethered away from the FET channel, or the charge packet 116 may be cleaved off into a released molecule 113. In either situation, there is less positive charge near the FET channel surface 69. This change in charge near the channel surface 69 is detectable as a change in conductance in the channel of the FET, and is directly correlated to the concentration of the target entity 118. For each embodiment of FIGS. 6-7, the described capture and dehybridization events cause a significant change in the charge of the linker probe 114 and/or in the electric double layer near the surface of the sensor 69. This change of charge can then be detected as a change in conductance of the FET sensor 71.

Another embodiment of this method is to modify the surface of the FET sensor with silanized/thiolated DNAzyme (DNA enzymes) or, alternatively, an aptamer/ssDNA molecule hybridized with a sacrificial complementary oligonucleotide, as linker probe molecule. As both types of probes are composed of oligonucleotides, each has a well known and fixed negative charge in solution. In the case of a DNAzyme, capture of target species catalyzes cleavage of the enzyme strand from the substrate strand of the molecule, causing a conductance change in the FET sensor proportional to charge of the enzyme strand. For the aptamer/ssDNA, the secondary hybridized sacrificial complementary oligonucleotide is detached upon capture of target species, causing a conductance change in the FET sensor proportional to charge of the sacrificial complementary oligonucleotide.

While various embodiments have been described above, they are presented by way of example only and are not to be construed as a limitation of the invention. Numerous changes to the disclosed embodiments can be made without departing from the scope of the invention. The scope of the invention is defined in accordance with the following claims and their equivalents.

What is claimed is:

1. A field effect transistor (FET) biosensor, comprising:
a) a field effect transistor having a FET gate dielectric surface;
b) a linker probe attached to said FET gate dielectric surface; wherein said linker probe comprises:
i) an enzymatic region capable of cleaving nucleic acids having a predetermined nucleic acid sequence, and
ii) an aptamer region attached to said enzymatic region, said aptamer region capable of selectively binding a target entity;
wherein binding of said target entity to said aptamer induces a measurable change in an electrical parameter of said FET.

2. The FET biosensor of claim 1, wherein said linker probe is a nucleic acid linker probe or a polypeptide nucleic acid linker probe.

3. The FET biosensor of claim 1, wherein said enzymatic region is a deoxyribozyme (DNAzyme) or a ribozyme.

5. The FET biosensor of claim 1, where said electrical parameter is conductance.

6. The FET biosensor of claim 1, wherein said linker probe further comprises a stump region coupled to said FET gate dielectric surface;
wherein said enzymatic region and said aptamer form a sacrificial region linked to said stump region and capable of detaching from said stump region in the presence of said target entity;
wherein binding of said target entity to said aptamer region results said enzymatic region cleaving nucleotides at said predetermined nucleic acid sequence, thereby releasing said sacrificial region from said stump region; and,
thereby inducing a measurable change in said electrical parameter of said FET.

7. The FET biosensor of claim 6, wherein said sacrificial region comprises a plurality of acid or base groups.

8. The FET biosensor of claim 6, wherein said stump region comprises a charge packet having little or no charge.

9. The FET biosensor of claim 6, where binding of said target entity to said aptamer region results in release of said cargo region from said linker probe.

10. The FET biosensor of claim 1,
wherein said enzymatic region and said aptamer region form a stump region attached at a first end to said FET gate electric surface;
wherein said FET biosensor further comprises a sacrificial region linked to said stump region at a second end, said sacrificial region capable of detaching from said stump region in the presence of said target entity;
wherein binding of said target entity to said aptamer region results in said enzymatic region cleaving nucleotides at said predetermined nucleic acid sequence, thereby releasing said sacrificial region from said stump region; and,
thereby inducing a measurable change in said electrical parameter of said FET biosensor.

11. The FET biosensor of claim 7, wherein said sacrificial region is further characterized as having a cargo region having a charge capable of being measured via said FET biosensor.

12. The FET biosensor of claim 1, wherein said target entity is a biological molecule.

13. The FET biosensor of claim 1 wherein said FET is an ion-sensitive field effect transistor (ISFET), a bio-FET, a nanowire FET, or a bio-finFET.

14. A field effect transistor (FET) biosensor, comprising:
a) a field effect transistor having a FET gate dielectric surface;
b) a linker probe attached to said FET gate dielectric surface; wherein said linker probe comprises:
i) a conformation changing region capable of changing three dimensional shape in the presence of a target entity;
ii) an aptamer region attached to said conformation changing region, said aptamer region capable of selectively binding a target entity;
wherein binding of said target entity to said aptamer region results in a conformational change of said conformation changing region, thereby inducing a measurable change in an electrical parameter of said FET.

15. The FET biosensor of claim 14, wherein said linker probe further comprises a cargo region charge carrier having a charge packet, whereby said conformation changing region positions said cargo region away from said FET gate dielectric surface when said target entity binds to said aptamer region, thereby inducing a measureable change in said electrical parameter of said FET.

16. The FET biosensor of claim 14, wherein said linker probe is characterized as having an oligonucleotide having:
a) a first strand of DNA;
b) a second strand of DNA;
c) a charge carrier cargo region; and
wherein said first strand of DNA hybridizes to said second strand of DNA in the absence of said target entity; and,
wherein in the presence of said target entity, said first strand dehybridizes from said second strand of DNA, and
wherein said cargo region charge carrier is at a further distance from said FET gate dielectric surface when said target entity is bound to said aptamer region compared to when said target entity is not bound to said aptamer, thereby inducing a measurable change in an electrical parameter of said FET when said target entity binds to said aptamer.

17. The FET biosensor of claim 16, wherein said cargo region is tethered to said FET gate dielectric surface in the presence of said target entity.

18. The FET biosensor of claim 16, further comprising an enzymatic region capable of cleaving linker probe in the presence of said target entity, resulting in the release said cargo region charge carrier in the presence of said target entity, thereby inducing a measurable change in an electrical parameter of said FET when said target entity binds to said aptamer.

19. A method of indirectly detecting the presence of or concentration of a target molecule with a FET sensor, comprising the steps of:
taking a first measurement of an electrical parameter of an FET to determine a baseline of said electrical parameter, said FET comprising
a) a field effect transistor having a FET gate dielectric surface;
b) a linker probe attached to said FET gate dielectric surface; wherein said linker probe comprises:
i) an enzymatic region capable of cleaving nucleic acids having a predetermined nucleic acid sequence, and
ii) an aptamer region attached to said enzymatic region, said aptamer region capable of selectively binding a target entity;
placing a solution having an unknown quantity of said target entity in contact with said linker probe on said gate dielectric surface;
taking a second measurement of said electrical parameter with said FET; and
determining the presence or concentration of said target molecule by comparing said first measurement of said electrical parameter with said second measurement of said electrical parameter,
whereby if said first measurement is different from said second measurement by a threshold value, said target molecule is determined to be present in the said solution; and,
whereby the greater the difference between said first measurement and said second measurement of said electrical parameter, the higher the concentration of said target molecule in the solution.

20. The method of claim 19, where said electrical parameter is drive current of said FET.
